(19) United States
(12) Patent Application Publication
(10) Pub. No.: US 2006/0211980 A1
(43) Pub. Date: Sep. 21, 2006

(54) Transdermal electrotransport drug delivery systems with reduced abuse potential

(76) Inventors: Michel J.N. Cormier, Mountain View, CA (US); Rama V. Padmanabhan, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/361,198

(22) Filed: Feb. 24, 2006

Related U.S. Application Data
(60) Provisional application No. 60/656,181, filed on Feb. 24, 2005.

(51) Int. Cl. A61N 1/30

(52) U.S. Cl. 604/20

(57) ABSTRACT

A transdermal electrotransport drug delivery system having reduced potential for abuse. The system uses ion barrier, preferentially ion exchange material to separate the drug from an antagonist and provides for the release of the antagonist with the agonist when the system is subject to abuse.

### CROSS REFERENCE TO RELATED U.S. APPLICATION DATA

The present invention claims the benefit under 35 USC 119(e) to U.S. Provisional patent application 60/656,181 filed on Feb. 24, 2005.

### TECHNICAL FIELD

The present invention relates to an electrotransport transdermal drug delivery system having reduced potential for abuse. In particular, the invention relates to a system for transdermal administration of cationic drugs, such as fentanyl and analogs thereof, to a subject through intact skin over an extended period of time, wherein the system provides for release of the antagonist to the drug when the dosage form (i.e. the transdermal drug delivery system) is subject to abuse.

### BACKGROUND

The transdermal administration of drugs with abuse potential, such as narcotic analgesics, i.e. opioids, for the treatment of both acute and chronic pain has been described in great detail. For example, the following patents: U.S. Pat. Nos. 4,466,953; 4,470,962; 4,588,580; 4,626,539; 5,006,342; 5,186,939; 5,310,559; 5,474,783; 5,656,286; 5,762,952; 5,948,433; 5,985,317; 5,958,446; 5,993,849; 6,024,976; 6,063,399 and 6,139,866 describe various ways of transdermally administering fentanyl and analogs thereof, such as alfentanil, carfentanil, lofentanil, remifentanil, sufentanil, trefentanil and the like, and are incorporated herein by reference. These patents disclose that fentanyl can be administered from a topically applied ointment, cream, or from a transdermal patch. Drugs can also be delivery transdermally by electrotransport, such as iontophoresis, electroporation, electroosmosis, etc. For example, U.S. Pat. Nos. 5,057,072; 5,322,502; 5,395,310; 6,049,722; and 6,216,033 are related to electrotransport transdermal delivery of drugs.

The potential for abuse of narcotic analgesics by intranasal, oral or parenteral routes is well known. Diversion and abuse of opioids may take several different forms. For example, the medication may be used by a person for whom it is not intended, i.e. diversion, or in amounts and/or frequency greater than prescribed, either by the originally prescribed route (e.g. oral or transdermal) or by an alternate route (e.g. parenteral, intravenous or intranasal). In order to prevent abuse of these substances, it has been proposed to provide dosage forms that combine the abusable substance with an amount of an antagonist for the abusable substance sufficient to eliminate the “high” associated with abuse of the substance without eliminating the other therapeutic benefits for which the drugs are intended to produce. See, for example, U.S. Pat. Nos. 3,773,955; 3,493,657; 4,464,378; 4,457,933; 4,626,539; 4,806,341; 4,935,428; 5,149,538; and 5,236,714; and International Publication No. WO 01/58451 A1, all of which are incorporated herein by reference. See also, Talwin; Levine J. D., et al, “Potentiation of pentazocine analgesia by low-dose naloxone”, J Clin Invest 1988; 82:1574-1577; Crain S M, Shen F-K, “Antagonist of excitatory opioid receptor function enhance morphine's analgesic potency and attenuate opioid tolerance/dependence liability”, Pain 2000; 84:121-131, which are incorporated herein by reference.

U.S. Pat. No. 5,236,714 describes transdermal dosage forms for delivering narcotic and psychoactive substances, the dosage form having a reduced potential for abuse. The transdermal dosage forms include an analgesic reservoir including a narcotic and an antagonist, and a releasing means through which the narcotic is released to the body. U.S. Pat. No. 5,149,538 describes a misuse-resistive dosage form for transdermal administration of opioids. The dosage form includes an opioid, an antagonist for the opioid that is releasable upon ingestion or solvent immersion, a barrier means separating the opioid from the antagonist and a delivery means for delivering the opioid.

Patent publication US20040013716 and WO03/090729 describe passive transdermal analgesic systems with reduced abuse potential. The transdermal dosage forms include an analgesic reservoir including a narcotic and an antagonist, which is released when the system is abused. Patent EP0781152B1 (stemmed from WO96/09850) is related to an electrotransport transdermal drug delivery system with reduced abuse potential by electronically limiting delivery to prevent unauthorized use.

Notwithstanding some success, the existing dosage forms have not been entirely satisfactory for reducing the potential for abuse, since the drug can be extracted from the dosage form for injection, inhalation or ingestion; or narcotic drug and antagonist may interact resulting in adverse physical and/or chemical interaction, such as undesirable ion permeation of the antagonist into the narcotic reservoir resulting in systemic delivery of the antagonist. In addition, the methods for reducing abuse of transdermal system with antagonist described thus far are specific to passive transdermal systems. Further, there has been no similar abusing reducing measures that have been applied to electrotransport systems, which are very different from passive transdermal systems, both in terms of structure, as well as how drugs are moved transdermally.

### SUMMARY

The present invention relates to an electrotransport transdermal drug delivery system having reduced potential for abuse. In particular, the invention relates to a system for electrotransport administration of a drug, e.g. a narcotic analgesic such as fentanyl or its salt, to a subject through intact skin over an extended period of time, wherein the system also contains an antagonist, which is not delivered to the patient under normal conditions of use but provides a deterrent against abuse of the system. The present invention has reduced potential for abuse, compared to systems without antagonists, without diminishing the therapeutic or beneficial effects of the drug (e.g. analgesic) when the system is applied to the skin, wherein the system provides no antagonist release or a substantially negligible release for minimized/negligible skin absorption of the antagonist during normal use. The transdermal drug delivery system of the present invention provides for release of the antagonist with the agonist when the system is subject to abuse. In another aspect, just the presence of the antagonist in the system or the packaging of the system provides a deterrent to abuse.

In one aspect, an ion exchange material is used to separate the antagonist from the drug. In another aspect, the ion exchange material separates the drug from any other adjacent reservoir containing chemical agents. In another aspect, an antagonist reservoir is in contact only with either an ion exchange membrane or solid material nonpermeable to the antagonist but to no other reservoir. In another aspect, the positively charged antagonist is present in the cathodic reservoir and is adjacent to skin or separated from the skin by an ion exchange membrane. In another aspect, a microporous membrane, such as a dialysis membrane, with a molecular weight cutoff smaller than the molecular weight of the antagonist is used instead of the ion exchange membrane.

In one aspect, the invention provides a transdermal electrotransport system for administering an analgesic through the skin. The system has a reduced potential for abuse of the analgesic and includes: electrode for conducting a current to drive analgesic ions of an ionizable analgesic; an analgesic reservoir having the analgesic ions; an antagonist source having an antagonist for the analgesic; and a barrier layer, the barrier layer separating the antagonist source from the analgesic reservoir, the barrier layer being substantially impermeable to the analgesic ions and to the antagonist, wherein the antagonist is released with the agonist when the system is subject to abuse. When the drug is ionic or the antagonist is ionic, preferably the barrier layer is an ion barrier, preferably an ion exchange barrier, such as an ion exchange membrane.

In another aspect, the invention provides a method for making a transdermal electrotransport system for administering an analgesic through the skin such that the system has a reduced potential for abuse of the analgesic. Steps in the method include: providing electrode for conducting a current to drive analgesic ions of an ionizable analgesic; providing ionic flow path from the electrode to an analgesic reservoir including the analgesic ions; and providing an antagonist source including an antagonist for the analgesic such that the antagonist source and the analgesic reservoir are separated by an ion barrier that is substantially impermeable to the analgesic ions and to the antagonist, wherein the antagonist is released when the system is subject to abuse.

In another aspect, the invention also provides a transdermal electrotransport system for administering an analgesic through the skin, the system including: anode portion having electrode for conducting a current to drive analgesic ions of a cationic analgesic and a analgesic reservoir having the cationic analgesic; cathode portion having an antagonist source including an antagonist for the analgesic and an ion barrier layer separating the antagonist source from skin of a user, the barrier layer being substantially impermeable to the analgesic and to the antagonist, wherein the antagonist is released with the agonist when the system is subject to abuse; wherein the cathode portion and the anode portion are visually indistinguishable from each other without referring to electrical connections thereto. In an embodiment, an ion barrier separates the agonist (drug) layer from another layer of ion source.

In one aspect, the present invention, using a barrier of ion exchange material to inhibit antagonist passage provides an advantageous way to block the antagonist while allowing ions of the opposite charge to that of the antagonist to pass through, thus allowing current to traverse through the electrode/reservoir portion to maintain efficiency of ion transport over an extended period of time, to effect drug delivery by electrotransport. This selective passage of ions of different polarity allows current to flow but blocks antagonist from passing. The use of ion exchange membrane to separate the drug reservoir from the antagonist reservoir works better than using mere nonion-exchange polymeric material for the separation because the repulsion by the immobile ions in the ion exchange material enables the repulsion of certain ions while allowing other desired ions to pass. Using ion exchange membrane, it is possible to prevent antagonist leakage to prevent substantial antagonist release so that there is minimized/negligible skin absorption of the antagonist during normal use.

### BRIEF DESCRIPTION OF THE FIGURES

The present invention is illustrated by way of example in embodiments and not limitation in the figures of the accompanying drawings in which like references indicate similar elements. The figures are not drawn to scale unless indicated otherwise in the content.

FIG. 1 illustrates a schematic, exploded view of a typical electrotransport device in which reservoir portions of the present invention can be used.

FIG. 2 illustrates a schematic, cross-section view through an embodiment of a subunit of a system this invention, including electrode/reservoir portions.

FIG. 3 illustrates a schematic, cross-section view through another embodiment of an electrode/reservoir portion of this invention.

FIG. 4 illustrates a schematic, cross-section view through an electrode/reservoir portion of another embodiment of this invention.

FIG. 5 illustrates a schematic, cross-section view through a particulate containing antagonist in another embodiment of this invention.

FIG. 6 illustrates a schematic, cross-section view through an electrode/reservoir portion of another embodiment of this invention.

FIG. 7 illustrates a schematic, cross-section view through an electrode/reservoir portion of yet another embodiment of this invention.

FIG. 8 illustrates a schematic, cross-section view through an anode/reservoir portion and cathode/reservoir portion of another embodiment of this invention.

FIG. 9 illustrates a schematic, cross-section view through an anode/reservoir portion and cathode/reservoir portion of yet another embodiment of this invention.

FIG. 10 illustrates a schematic, cross-section view through an anode/reservoir portion and cathode/reservoir portion of another embodiment of this invention.

### DETAILED DESCRIPTION

Overview:

The present invention is directed to an electrotransport transdermal drug delivery system having reduced potential for abuse, without diminishing the therapeutic or beneficial effects of the analgesic when the system is applied to the skin. In particular, the system of the present invention provides ion barriers controlling or preventing release of an antagonist such that any release of the antagonist, if any, under normal prescription use would be negligible. The antagonist is released with the agonist when the dosage form is subject to abuse. The system provides for a substantially minimized/negligible skin absorption of the antagonist during normal use.

The practice of the present invention will employ, unless otherwise indicated, conventional methods used by those in pharmaceutical product development within those of skill of the art. Such techniques are explained fully in the literature. See, e.g. Gale, R., Chandrasekaran, S. K., Swanson, D. and Wright, J., “Use of Osmotically Active Therapeutic Agents in Monolithic Systems” J. Membrane Sci., 7 (1980), 319-331; Patini, G. A. and Chein, Y. W., Swarbrick, J. and Boylan, J. C., eds, Encyclopedia of Pharmaceutical Technology, New York: Marcel Dekker, Inc., 1999 and Gale, R., Hunt, J. and Prevo, M., Mathiowitz, E., ed, Encyclopedia of Controlled Drug Delivery Patches, Passive, New York: J Wiley & Sons, Inc, 1999.

Definitions:

In describing the present invention, the following terminology will be used in accordance with the definitions set out below.

The singular forms “a,”, “an” and “the” include plural referents unless the context clearly dictates otherwise. Thus, for example, reference to “a polymer” includes a single polymer as well as a mixture of two or more different polymers, reference to “a permeation enhancer” includes a single permeation enhancer as well as two or more different permeation enhancer in combination, and the like.

As used herein, the term “an analog of fentanyl” (hereafter referred to as “analog”) refers to potent and effective analgesics such alfentanil, carfentanil, lofentanil, remifentanil, sufentanil, trefentanil, and the like.

As used herein, the term “antagonist” refers to a substance that diminishes or prevents the action of a chemical agent. An antagonist refers to either an uncharged form or a salt thereof, e.g. HCl, HBr, citrate salt, etc. unless specified to be otherwise in context. Similarly, “agonist” can refer to an uncharged form of a drug or a salt thereof, e.g. HCl, HBr, citrate salt, etc. unless specified to be otherwise in context.

As used herein, the term “substantially prevents release of the antagonist from the system” implies a transdermal drug delivery system wherein the amount of antagonist that is released from the system in normal use or upon casual contact or incidental exposure to water does not substantially reduce the therapeutic (e.g. analgesic) effect of the drug in the transdermal drug delivery system. “Analgesic effect”, as used in this context, is meant to refer to therapeutic and/or pharmacokinetic effects, as determined by any conventional clinical, in vitro, in vivo, pharmacokinetic, or pharmacodynamic method.

The term “substantially prevents release of the antagonist from the system”, as used herein additionally or alternatively implies a transdermal analgesic system wherein minimal amount of antagonist is released from the system upon casual contact or incidental exposure to water, such that there is minimal or negligible antagonist skin absorption of the antagonist during normal use.

Illustratively, the term “substantially prevents release of the antagonist from the system” is meant to encompass transdermal analgesic systems wherein the total amount of antagonist that is released from the system in normal use or upon casual contact or upon incidental exposure to water divided by the amount of drug that is released from the system under the same conditions is less than 20%, such as less than about 20%, less than 10%, less than about 10%, less than 5%, less than about 5%, less than 2%, less than about 2%, less than 1%, less than about 1%, about zero, and/or zero.

As used herein, the term “incidental exposure to water” refers to short-term exposure to high humidity or brief exposure to liquid water, such as during showering, sweat, and the like.

As used herein, the term “component” refers to an element within the analgesic reservoir, including, but not limited to, an analgesic as defined above, additives, permeation enhancers, stabilizers, dyes, diluents, plasticizer, tackifying agent, pigments, carriers, inert fillers, antioxidants, excipients, gelling agents, anti-irritants, vasoconstrictors and the like.

As used herein, the term “antagonist release when abused” refers to exposure of the antagonist to solvent and extraction of the antagonist with the agonist when the drug delivery system is subject to abuse. The solvent can be a body fluid, such as saliva. The substantial prevention of antagonist release during normal subscription use substantially minimizes skin absorption of the antagonist.

As used herein, the term “abuse of a transdermal drug delivery system” refers to the use of a transdermal drug delivery system other than as indicated by the product labeling, including tampering or misusing the system, subjecting the system to diversion, ingestion or substantial immersion of the system in a solvent for intravenous administration, buccal administration, and the like.

Some of the systems described will be more resistant to water than others. Also, some systems will be more abusable than others. Certain determined individuals will abuse a system even though the process required to do so is highly complex, while many others will abuse a particular system only if the task is easy and risk free. For many potential abusers, merely knowing that there is an antagonist incorporated in the system will prevent them for abusing the system because of the complexity of the task and that other alternative substances of abuse are available. Inclusion of an antagonist in the packaging or in the circuit board housing will prevent these less determined individuals from abusing the system. Therefore, the goal is not to prevent abuse completely for all potential abusers, but to reduce abuse and to make it unpalatable to casual users, which could be perhaps a large percentage of potential abusers.

### Modes of Carrying Out the Invention

The present invention provides an analgesic system for transdermal delivery of ionizable drugs by electrotransport, such as iontophoresis, with release of antagonist to the drug when the system is subject to abuse. In an aspect, the present invention uses ion exchange material to separate the drug ions from the antagonist. In an aspect, the ionizable drug includes salt of fentanyl, analogs thereof, or a combination thereof. The analgesic drug is delivered for analgesic purposes to a subject through intact skin over an extended period of time. The system has reduced potential for abuse and a substantially minimized/negligible skin absorption of the antagonist during normal use. In particular, the transdermal drug (e.g. analgesic such as fentanyl or analog thereof) system of the present invention provides for the release of the antagonist with the agonist when the system is subject to abuse. Because of the differences in potency of the different agonist and antagonists, the different doses of agonist given to different patients, and the differences in sensitivity of different patients, precise definition of the maximum amount of antagonist that can cross the skin without affecting the efficacy of the agonist during normal prescription use can vary depending on the selection of agonists and antagonists. As a general rule, preferably less than 0.5 mg, more preferably less than 0.1 mg, of antagonist should be delivered into the systemic circulation over a period of 8 hours and more preferably 24 hours.

Because of the differences in potency of the different agonists as well as differences in potency of the different antagonists, the precise ranges of concentrations for the agonist and the antagonist can vary depending on the selection of agonists and antagonists. Similarly, the ratio of the antagonist to the agonist can also vary depending on the agonist and antagonist selection. As a general rule, to be effective, the concentration of the agonist in the agonist reservoir is preferably between 0.05 wt % and 20 wt %, more preferably between 0.1 wt % and 10 wt %. Similarly, the concentration of the antagonist in the antagonist reservoir is preferably between 0.05 wt % and 20 wt %, more preferably between 0.1 wt % and 10 wt %.

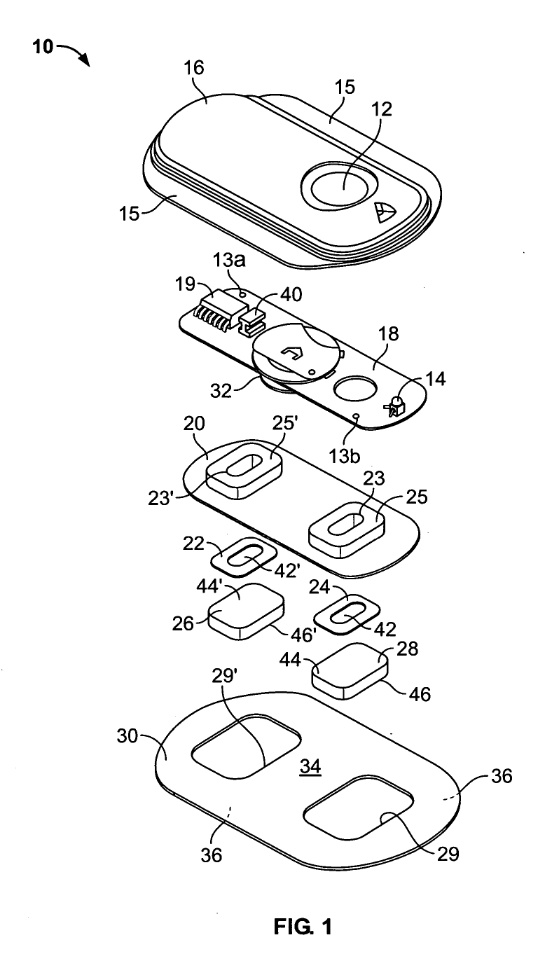

Electrotransport devices, such as iontophoretic devices are known in the art, e.g. U.S. Pat. No. 6,216,033, and can be adapted to function with antagonist separation of the present invention. A typical iontophoretic transdermal device that can be so adapted is shown in FIG. 1 and described in the following. FIG. 1 depicts an exemplary electrotransport device that can be used in accordance with the present invention. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable elastomer (e.g. ethylene vinyl acetate).

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′,29 in adhesive 30.

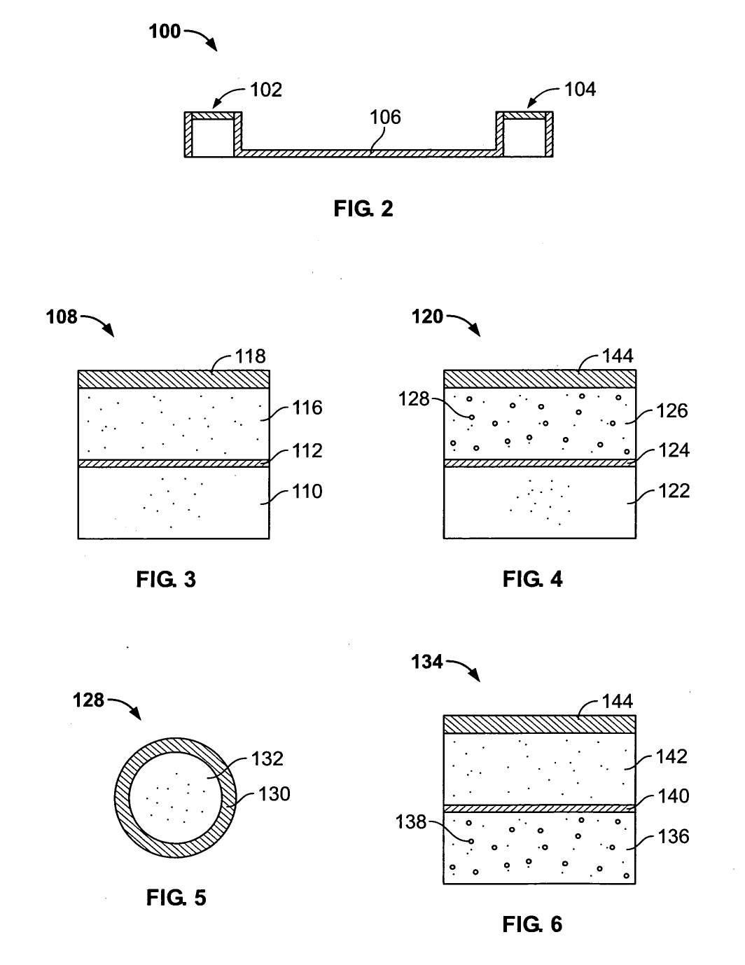

When using an ion exchange membrane to separate an ionic drug from an antagonist, in some embodiments, it is preferred that a layer containing the antagonist contact only and bordered by either the ion exchange membrane or other material nonpermeable to liquid (such as solid plastic housing, electrode, etc.) by the antagonist so as to prevent undesirable migration of the antagonist. FIG. 2 shows a schematic view of an illustrative subunit of an electrotransport drug delivery system with both anodic and cathodic electrode and reservoir containing portions (“electrode/reservoir portions”) of an embodiment of an eletrotransport system of the present invention. The subunit 100 includes anode/reservoir portion 102 and cathode/reservoir portion 104 connected and supported by housing portion 106. Housing portion 106 can be adapted into housing 20 of FIG. 1 or other similar electrotransport devices. FIG. 3 shows an embodiment of an electrode/reservoir portion. The electrode/reservoir portion 108 is for delivering an ionic drug. The electrode/reservoir portion 108 includes a drug reservoir 110 in layer form that is to be disposed proximate to or on the skin of a user for delivery of drug to the user. The drug reservoir 110 includes an ionizable drug. To be deliverable by electrotransport such as iontophoresis, at least some of the ionizable drug would be in ionic form. An ion rejection membrane, such as an ion exchange membrane 112 separates and isolates an antagonist reservoir 116 from the drug reservoir 110. The antagonist reservoir 116 is in layer form and contains an antagonist to the drug in the drug reservoir 110. The ion exchange membrane contains charges that are of the same polarity as ions of the antagonist so that the antagonist ions are repelled by the ion exchange membrane. An electrode layer 118 contacts the antagonist reservoir to provide an electric current to drive the drug ions in the drug reservoir 110 further away from the electrode 118 to the skin. Both the antagonist reservoir and the drug reservoir 110 are of compositions that allow ions to traverse therethrough under an electromotive force provided by the electrode 118. The antagonist reservoir 116 is surrounded either by the ion exchange membrane 112, or a solid nonpermeable material, such as the electrode 118 or part of the housing (shown in FIG. 2). As a more specific embodiment, the drug is a cationic drug such as a salt of fentanyl, the ion exchange membrane 112 is an anion exchange membrane, preferably presenting a strong base anionic functionality, the electrode is made of silver, and the antagonist is a cationic antagonist. In some preferred embodiments, the electrode in contact with the antagonist reservoir is porous or contains discrete gaps or holes to facilitate extraction of the antagonist during certain modes of abuse of the system (i.e. whole system extraction in water).

It is to be understood that the structures of FIG. 3 can be adaptable for a cathode/reservoir portion in which the drug is an anionic drug, the ion exchange membrane is a cation exchange membrane, and the antagonist is an anionic antagonist. In such a case, the electrode can be more preferably made of silver chloride. In the description of electrode/reservoir portions, it is to be understood that where an anode/electrode is described for delivery of a cationic drug, the structure can be adapted similarly for delivery of an anionic drug by replacing structures and elements of an ionic property (e.g. cationic) with structures of opposite ionic property (e.g. anionic).

FIG. 4 shows another embodiment of an anode/reservoir portion 120. Again, a drug reservoir 122 contains a cationic drug, such as a fentanyl salt, e.g. fentanyl HCl, and is to be placed proximate or on the skin of a user for transdermal electrotransport drug delivery. More distal from the skin, an anion exchange membrane 124 separates and isolates a antagonist containing-layer 126 containing particulates 128, which include an antagonist, preferably a positively charged antagonist, of the cationic drug. Still more distal to the skin is an electrode, preferably silver, for contacting the antagonist containing-layer 126 to provide electromotive force for driving the cationic drug from the drug reservoir 122 to the skin. As shown in FIG. 5, the particulate 128 includes antagonist material 132 encapsulated within a layer 130 of anion exchange material such that positively charged antagonist cannot pass therethrough, or if there is any leak, the leakage would be not significant and negligible skin absorption of the antagonist would occur.

With an ion exchange membrane 130 protecting the drug reservoir from the antagonist, an alternative wherein the particulate is sealed with a nonionic material can also be practiced. One or both of the anion exchange material 124, 130 shown in FIG. 4 and FIG. 5 can be replaced with a nonion-exchange polymeric layer that is capable to isolate the antagonist under normal use and yet releases the antagonist when the system is under abuse, i.e. placed in nonprescription, illicit use, such as being placed in a solvent, or chewed.

FIG. 6 shows yet another embodiment of an anode/reservoir portion. The anode/reservoir portion 134 has a drug reservoir 136 containing a cationic drug and is to be placed proximate or on the skin. Dispersed in the drug reservoir 136 are antagonist-containing particulates 138, which include an antagonist, preferably a positively charged antagonist, of the cationic drug. The particulates 138 can be similar to the particulates 128 of FIG. 4 and FIG. 5. More distal from the skin, an anion exchange membrane 140 separates and isolates a reservoir 142 containing a source of halide ions, such as chloride or bromide ions. Still more distal to the skin is a silver electrode 144 for contacting the reservoir 142 of halide ion source to provide electromotive force for driving the cationic drug from the drug reservoir 122 to the skin.

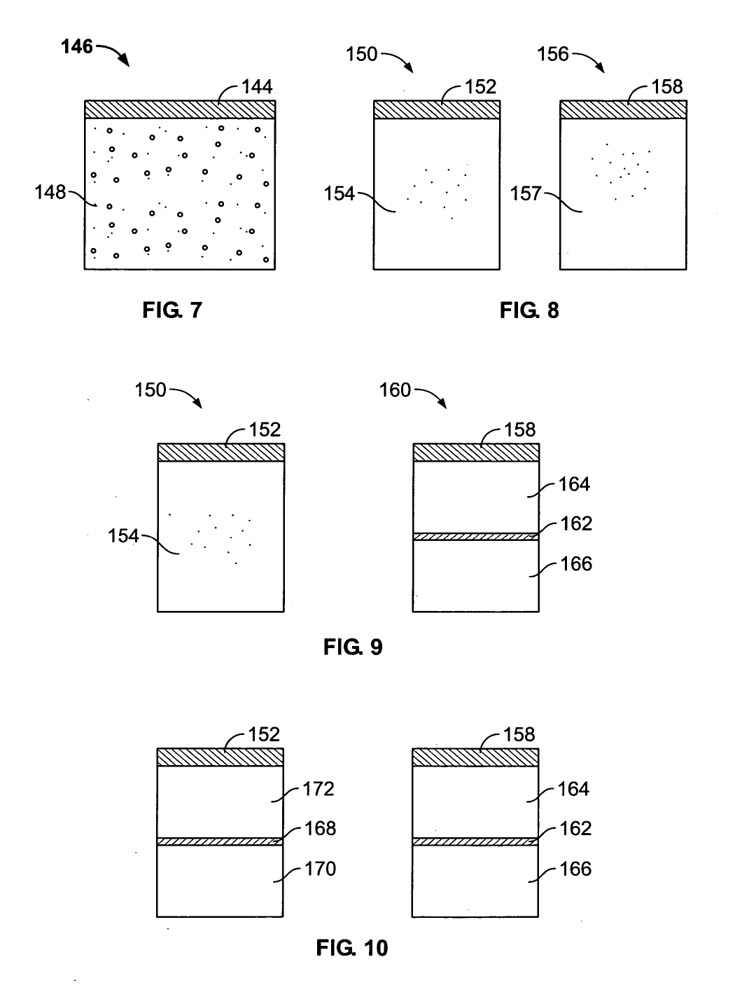

FIG. 7 shows yet another embodiment of an anode/reservoir portion. In this embodiment there is only one reservoir layer. The anode/reservoir portion 146 has a drug reservoir layer 148 containing a cationic drug and is to be placed proximate or on the skin. Dispersed in the drug reservoir layer 148 are antagonist-containing particulates 150, which include an antagonist, preferably a positively charged antagonist, of the cationic drug. The particulates can be similar to the ones described for FIG. 4 and FIG. 5. More distal to the skin is a silver electrode 144 for contacting the reservoir 148.

The drug reservoir can be formed of any material as known in the prior art suitable for making drug reservoirs. The reservoir formulation for transdermally delivering the cationic drugs by electrotransport is preferably composed of an aqueous solution of a water-soluble salt such as HCl or citrate salts of a cationic drug, such as fentanyl or sufentanil. More preferably, the aqueous solution is contained within a hydrophilic polymer matrix such as a hydrogel matrix. The drug salt is present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the analgesic reservoir formulation includes at least 30 wt % water during transdermal delivery of the drug.

Although not critical to this aspect of the present invention, the applied electrotransport current density is typically in the range of about 25 to 150 μA/cm² and the applied electrotransport current is typically in the range of about 75 to 500 μA.

The anodic drug salt-containing hydrogel can suitably be made of a any number of materials but preferably is composed of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer, a buffer, a humectant, a thickener, water and a water soluble drug salt (e.g. HCl salt of the drug). A preferred hydrophilic polymer matrix is polyvinyl alcohol such as a washed and fully hydrolyzed polyvinyl alcohol (PVOH), e.g. Mowiol 66-100 commercially available from Hoechst Aktiengesellschaft. A suitable buffer is an ion exchange resin which is a copolymer of methacrylic acid and divinylbenzene in both an acid and salt form. One example of such a buffer is a mixture of Polacrilin (the copolymer of methacrylic acid and divinylbenzene available from Rohm & Haas, Philadelphia, Pa.) and the potassium salt thereof. A mixture of the acid and potassium salt forms of Polacrlin functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. Methocel K100MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

In one preferred embodiment, the anodic drug salt-containing hydrogel formulation includes about 10 to 15 wt % polyvinyl alcohol, 0.1 to 0.4 wt % resin buffer, and about 1 to 30 wt %, preferably 1 to 2 wt % drug salt, preferably the hydrochloride salt, for example, hydrochloride of fentanyl or sufentanil. The remainder is water and ingredients such as humectants, thickeners, etc. The polyvinyl alcohol (PVOH)-based hydrogel formulation is prepared by mixing all materials, including the drug salt, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for fentanyl electrotransport.

In a preferred embodiment, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

For more effective delivery by electrotransport such as iontophoresis, salts of such analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In the case of counterions bearing more than one negative charge, the drug can be added in excess of the acidic counterion. For example, the citrate salt of fentanyl can be the monocitrate or the hemicitrate.

In one embodiment of the invention, the drug reservoir includes at least one buffer for controlling the pH in the drug reservoir. For a cationic drug, such as fentanyl salt, examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine, dipeptides such as Gly-Asp, Asp-His, His-Glu, or mixtures thereof.

The pH of the drug reservoir is kept at a level that provides the drug in ionic form so as to be repelled by ion exchange barrier, preferably ion exchange membrane, and yet at a level that is compatible to the skin, i.e. that can enable therapeutic migration of the drug ions without causing unacceptable irritation or reaction from the skin. Preferably, the pH of the cationic drug reservoir formulation, e.g. narcotic analgesic agent, is below approximately pH 6. More preferably, the pH of the drug reservoir, preferably for narcotic analgesic agent formulation, is in the range of approximately pH 2-6. Even more preferably, the pH of the narcotic analgesic agent formulation is in the range of approximately pH 3-5. At such pH ranges, the cationic drug, e.g. fentanyl or sufentanil will be in salt or ionic form rather than in the base form and yet be compatible to the skin. Biasing the drug to the ionic form rather than base form prevents undesirable amount of base drug from crossing the anion exchange membrane.

In one embodiment of the invention, the narcotic analgesic formulation includes at least one biocompatible halide source, such as a bromide or a chloride source, e.g. the hydrochloride salt of the antagonist or the chloride form of an anionic exchange resin such as cholestyramine. Other resins that can release chloride or bromide ions may also be used. The halide ions can react with the silver ions that form at oxidation of silver at the anode as the anode provides positive charges for driving the cationic drug to the skin, thereby forming insoluble silver halide, e.g. AgCl, that will participate, thus removing excessive silver ions and preventing them from accumulating or traveling to the skin. Other components, such as permeation enhancers can be present in the drug reservoir. Permeation enhancers are known to persons skilled in the art of electrotransport transdermal drug delivery, for example, for the delivery of cationic drugs such as fentanyl and analogs thereof. In some cases, permeation enhancers can be used in the drug reservoir up to 25 wt % or more.

In a preferred embodiment, the analgesic reservoir is essentially dry during storage and is rehydrated at the time of use. This can be achieved as disclosed in U.S. Pat. Nos. 5,582,587, 5,533,972, 5,385,543, 5,320,598, 5,310,404, and 5,288,289. The matrix of the agonist, antagonist and salt reservoirs can be any material adapted to absorb and hold a sufficient quantity of liquid therein in order to permit transport of agent therethrough by iontophoresis. For example, gauzes made of cotton or other absorbent fabrics as well as pads and sponges, both natural and synthetic, may be used. More preferably, the matrix of the reservoirs is composed, at least in part, of a hydrophilic polymer material. Most preferably, the matrix of the reservoirs is a solid polymer matrix composed at least in part of a hydrophilic polymer. Both natural and synthetic hydrophilic polymers may be used. Suitable hydrophilic polymers include copolyesters such as Hytrel® sold by DuPont de Nemours & Co. of Wilmington, Del., polyvinylpyrrolidones, polyvinyl alcohol, polyethylene oxides such as Polyox.RTM. manufactured by Union Carbide Corp., Carbopol® manufactured by BF Goodrich of Akron, Ohio; blends of polyoxyethylene or polyethylene glycols with polyacrylic acid such as Polyox® blended with Carbopol®, polyacrylamide, Klucel®, cross-linked dextran such as Sephadex (Pharmacia Fine Chemicals, AB, Uppsala, Sweden), Water Lock® (Grain Processing Corp., Muscatine, Iowa) which is a starch-graft-poly(sodium acrylate-co-acrylamide) polymer, cellulose derivatives such as hydroxyethyl cellulose, hydroxypropylmethylcellulose, low-substituted hydroxypropylcellulose, and cross-linked Na-carboxymethylcellulose such as Ac-Di-Sol (FMC Corp., Philadelphia, Pa.), hydrogels such as polyhydroxyethyl methacrylate (National Patent Development Corp.), natural gums, chitosan, pectin, starch, guar gum, locust bean gum, and the like, along with blends thereof. Of these, polyvinylpyrrolidones are preferred.

Optionally, the matrix of the reservoirs may also contain a hydrophobic, preferably heat fusible, polymer in order to enhance the lamination of reservoir layers to the adjacent layers (e.g. insulators, electrodes, ionic exchange membrane, and any other optional membrane and/or adhesive layers) and the cohesion of these layers making it difficult to abuse the system by attempting to separate the agonist reservoir. Suitable hydrophobic polymers for use in the matrix of the reservoirs include, without limitation, polyethylene, polypropylene, polyisoprenes and polyalkenes, rubbers, copolymers such as Kraton®, polyvinylacetate, ethylene vinyl acetate copolymers, polyamides such as Nylon, polyurethanes, polyvinylchloride, acrylic or methacrylic resins such as polymers of esters of acrylic or methacrylic acid with alcohols such as n-butanol, n-pentanol, isopentanol, 2-methyl butanol, 1-methyl butanol, 1-methyl pentanol, 2-methyl pentanol, 3-methyl pentanol, 2-ethyl butanol, isooctanol, n-decanol, or n-dodecanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tert-butylacrylamide, and itaconic acid, N-branched alkyl maleamic acids wherein the alkyl group has 10-24 carbon atoms, glycol diacrylates, and blends thereof. Most of the above listed hydrophobic polymers are heat fusible. Of these, ethylene vinyl acetate copolymers are preferred. When the drug or electrolyte is present in the reservoir matrix before hydration, blending of the drug or electrolyte with the hydrophilic polymer matrix components can be accomplished mechanically, either by milling, extrusion or hot melt mixing, for example.

The resulting reservoir layers may then be prepared by solvent casting, extrusion or by melt processing, for example. In addition to the drug and electrolyte, the reservoirs may also contain other conventional materials such as dyes, pigments, inert fillers, and other excipients. In some preferred embodiments, the electrode in contact with the antagonist reservoir is porous or contains discrete gaps or holes to facilitate extraction of the antagonist during certain modes of abuse of the system (i.e. whole system extraction in water).

An antagonist reservoir (or layer) can also be made with a matrix or gel material that is similar to those for making the drug reservoir. Similar to the matrix or gel for the drug reservoir, a hydrogel is preferred as a material for making the antagonist reservoir. Formulation for a hydrogel that can be used for the antagonist reservoir, for example, can include about 10 to 30 wt % polyvinyl alcohol, about 0.1 to 0.4 wt % resin buffer, and about 40 to 90 wt %, more preferably about 50 to 85 wt % water. Ingredients such as humectants, thickeners, etc., can also be included. The polyvinyl alcohol (PVOH)-based hydrogel formulation is prepared by mixing all materials in a single vessel at elevated temperatures of about 90° C. to 95° C. for at least about 0.5 hour. The hot mix is then poured into foam molds and stored at freezing temperature of about −35° C. overnight to cross-link the PVOH. Upon warming to ambient temperature, a tough elastomeric gel is obtained. Other reservoir layers, such as the reservoir 142 containing halide ion source can be made with hydrogel in a similar way by one skilled in the art.

The antagonist reservoir can also be composed of a matrix having hydrophobic polymers as long as there are hydrophilic materials, such as ionic resins, in the composition to allow current to flow. Similarly, the drug reservoir or other reservoir for containing other ions or components can also be made with matrix containing hydrophobic material so long as hydrophilic materials are included in the matrix for allowing electrical current flow.

In certain aspects, an ion exchange barrier substantially prevents the antagonist, as well as the drug from crossing from their respective reservoir to the other. In an anion exchange membrane, the fixed positively charge is bound to the membrane and will inhibit positively charged ions from passing through the membrane while allowing negatively charged ions to pass. A cation exchange membrane repels negatively charged ions while allows positively charged ions to pass. Although it is desirable that the ion exchange membrane blocks the drug ions and the antagonist ions, it is important that that ion exchange membrane allows ions of charge opposite to that of the drug ion to cross so that current flow is enabled to maintain the electrotransport process and that the device can be used over a period of time, preferably up to a day or more. In some instances, an ion exchange membrane or coating that is not molecularly tight can be used in which nonionized drug molecules (e.g. fentanyl base), or even nonionized antagonist molecules (e.g. naltrexone base) may pass through the ion exchange membrane or coating. In such instances, the ionic nature of the ionic exchange membrane will prevent the drug ion or antagonist ion to pass. Thus, keeping the drug reservoir and the antagonist reservoir at the proper pH at which substantially all of the antagonist, and preferably also the drug, is in ionic form is preferred.

Preferably, the pH of the antagonist formulation is such that the antagonist is almost completely in the ionized state. Indeed in the absence of an electric field that drives the ion towards the skin, the skin permeability of ionized species is much lower than that of the neutral or non-ionized form of the molecule. This poor skin permeability of the charged antagonist allows the antagonist reservoir to be positioned directly in contact with the skin in the embodiments where the charged antagonist is in the drug reservoir, e.g. positively charged antagonist is present in the cathode reservoir. In the embodiments where the antagonist is separated from the agonist reservoir by an ionic exchange membrane, it is also preferred that most of the antagonist is ionized, since non-ionized molecules can freely go through ion exchange membranes. The fractions of charged and uncharged species of the antagonist are directly related to the pKa of the antagonist and the pH of the formulation. Most of the antagonists for fentanyl and analogs thereof are present at a basic pKa in the range 8 to 9.5 (exception is amiphenazole with a pKa of about 7). About two pH units below the pKa, 99 percent of the narcotic antagonist molecules are positively charged. About three pH units below the pKa, 99.9 percent of the molecules are positively charged. Preferably the pH of the antagonist reservoir formulation is at least 2 pH units below the pKa of the narcotic antagonist, more preferably 3 pH units below the pKa. Even more preferably, to be compatible with skin and not significantly affecting the pH of the drug reservoir, the pH of the antagonist reservoir formulation is near to that of the drug reservoir. The pH of the antagonist formulation is preferably below approximately pH 6. More preferably, the pH of the antagonist formulation is in the range of approximately pH 2-6. Even more preferably, the pH of the antagonist formulation is in the range of approximately pH 3-6. Similar reasoning can be applied to other drugs and antagonists.

When the agonist is a narcotic analgesic, the antagonists are preferably chosen from the group consisting of the narcotic antagonists, preferably in salt form, such as HCl, HBr, citrate, etc. Preferred compounds include amiphenazole, cyclazocine, levallorphan, methylnaltrexone, nadide, nalmefene, naloxone, nalorphine, nalorphine dinicotinate, and naltrexone. Alternatively, the antagonist is chosen from the group of partial agonist-antagonists. Preferred compounds include butorphanol, nalbuphine, noscapine, and pentazocine. Such antagonists are ionic, positively charged at the relevant acidic pH.

When the drug is cationic, e.g. fentanyl or sufentanil salts, the applicable ion exchange material is an organic resin with pendent cationic groups (e.g. cholestyramine resin sold by Rohm & Haas, Philadelphia, Pa. Other applicable chloride resins are cross-linked acrylic resins such as Macroprep High Q Support resin sold by Bio-Rod Laboratories, Richmond, Calif.; cross-linked polystyrene resins such as Cholestramine resin, Duolite A-7 resin, Amberlite IRA-68 and IRA-958 resins, all sold by Rohm & Haas, Philadelphia, Pa.; and epichlorohydrin/tetraethylenetriamine resins such as Colestipol sold by the Upjohn Co., Kalamazoo, Mich. Such ion exchange material can be made into a membrane, film, sheet, coating, or a deposit on particles such as particles that contain an antagonist for the drug to be delivered.

When the drug is anionic, then the ion exchange material also is an organic resin with pendent anionic groups (e.g. Amberlite IRP-69 sulfonated copolymer of styrene and divinyl benzene commercially available from Rohm & Haas Corporation, Philadelphia, Pa.). Cation exchange material can also be made into a membrane, film, sheet, coating, or as a deposit on particles containing an antagonist for the anionic drug.

Examples of anionic and cationic selective membranes are described in the article “ACRYLIC ION-TRANSFER POLYMERS”, by Ballestrasse et al, published in the Journal of the Electrochemical Society, November 1987, Vol. 134, No. 11, pp. 2745-2749. An additional appropriate anion exchange membrane would be a copolymer of styrene and divinyl benzene reacted with trimethylamine chloride to provide an anion exchange membrane (see “Principles of Polymer Systems” by F. Rodriguez, McGraw-Hill Book Co., 1979, pgs 382-390). These articles are incorporated herein by reference in their entirety. For anionic exchange membranes, strong base anionic functionality (such as styrene quaternary ammonium type anion-exchange resin) is particularly desired, while for cationic exchange membranes strong acid cationic functionality (such as styrene sulfonic acid type cation-exchange resin) is particularly desired for their high permselectivity. The resins are usually blended with a polymer, such as polyethylene, and other additives to manufacture the ionic exchange membrane. Such membranes can be obtained through suppliers such as Sybron Chemicals Inc. An additional appropriate cationic permeable material for use in conjunction with a negatively charged drug or antagonist would be a sulfonated styrene polymer or a sulfonated fluorocarbon polymer, e.g. Nafion™.

One skilled in the art will be able to make the electrode/reservoir portions of the present invention using appropriate ion exchange membranes.

In order to appropriately release the antagonist during some modes of abuse of the system (i.e. attempt to isolate the agonist reservoir from the system), the membrane is preferably constructed with weak spots so that they easily rupture, break or leak during such attempt (as compared to other areas that are not as weak). For example, at the weak spots of the membrane the barrier is more easily ruptured so that when the subject attempts to abuse the drug delivery system, e.g. by using stress or solvent, the weak spot would preferentially break and allow the antagonist to contaminate the agonist reservoir. However, when the system is in normal prescription use, the weak areas will have sufficient integrity to prevent antagonist release so as to result in negligible skin absorption of the antagonist during normal use. There are many ways that weak spots can be generated. For example, thinner areas can be easily generated during the manufacturing process. Thinner spots would not prevent ionic migration as effectively as the rest of the membrane but these thinner spots may represent a very small fraction of the total area of the ionic membrane, and therefore result in negligible diffusion of the antagonist across the membrane and into the agonist or the salt reservoir adjacent to it. Distribution of the thinner areas along a line, for example (in much the same way as a perforated sheet of paper for easy tear off) results in a membrane that would rupture if there is any attempt to isolate the agonist reservoir. In addition, the membrane may contain physical extensions into the agonist and/or antagonist reservoir to further prevent attempts by a potential abuser to isolate (or remove) the agonist reservoir without contamination with some antagonist. One skilled in the art will be able to make the electrode/reservoir portions of the present invention using appropriate ion exchange membranes.

As mentioned before, in certain embodiments a polymeric nonion exchange barrier can be used to isolate the antagonist. For example, a nonionic microporous membrane (such as a dialysis membrane) with a molecular weight cutoff smaller than the molecular weight of the antagonist is used instead of the ion exchange membrane. Typically, the molecular weight of the antagonist is at least about 200 Da, and generally more than 300 Da. Preferably, the molecular weight cutoff of the membrane should be 300 or less, more preferably, 200 or less, even more preferably 100 or less. Because the molecular weight of the agonist drug (e.g. fentanyl or analog thereof) is also at least about 200 Da, and generally more than 300 Da, the same dialysis membrane will also inhibit diffusion of the agonist into the antagonist reservoir. This embodiment can also be practiced when the antagonist is in the form of a particulate. Further, when the dialysis membrane is used as a barrier separating the agonist compartment from the antagonist compartment, the molecular weight cutoff of the dialysis membrane should be large enough to allow small ions to pass through, thus allowing current to traverse through the electrode/reservoir portion to maintain efficiency of ion transport over an extended period of time, to allow delivery of the agonist by electrotransport. This selective passage of small inorganic ions of different polarity allows current to flow but blocks antagonist and agonist from passing. The molecular weight of the small inorganic ions is generally less than 100 and usually less than 50. Preferably, the molecular weight cutoff of the membrane should be between about 50 and 300, more preferably between about 50 and 200. Using dialysis membranes, it is possible to prevent antagonist leakage to prevent substantial antagonist release so that there is minimized/negligible skin absorption of the antagonist during normal use. Examples of materials used to manufacture dialysis membrane include collodion, cellophane, cellulose, regenerated cellulose, cellulose esters, such as cellulose acetate, other cellulose derivatives, cuprophan, and polysulfone-based polymers. The manufacture of dialysis membranes of various molecular weight cutoffs are known in the art.

A suitable electrotransport device includes an anodic donor electrode, preferably made of silver, and a cathodic counter electrode, preferably made of or including silver chloride. The donor electrode is in electrical contact with the donor reservoir containing the aqueous solution of a salt of a cationic drug, e.g. fentanyl salt or sufentanil salt. As described above, the donor reservoir is preferably a hydrogel formulation. The counter reservoir also preferably includes a hydrogel formulation containing a (e.g. aqueous) solution of a biocompatible electrolyte, such as citrate buffered saline. The anodic and cathodic hydrogel reservoirs preferably each have a skin contact area of about 1 to 5 cm² and more preferably about 2 to 3 cm². The anodic and cathodic hydrogel reservoirs preferably have a thickness of about 0.05 to 0.25 cm, and more preferably about 0.15 cm. The applied electrotransport current is about 150 μA to about 240 μA, depending on the analgesic effect desired. Most preferably, the applied electrotransport current is substantially constant DC current during the dosing interval.

In some instances it may be preferable to further separate the agonist from the antagonist to avoid possible mixing of the agonist and antagonist during storage, transport, handling, and administering the drug delivery system to the patient and to reduce the potential of abuse. One way is to make the location of the agonist difficult to identify. In order to extract the agonist the abuser would have to extract both anodes and cathode gels or the whole system. Thus, it provides advantages to locate the antagonist outside the electrode/reservoir portion at which the drug reservoir is located.

In a preferred embodiment, the system is made in a way the anodic and the cathodic sides of the electrotransport system are not readily visually distinguishable without referring and tracing back the electrical connection to find out which is anode and which is cathode. The system geometry would preferably be symmetrical with regard to the axis between anode/reservoir portion and cathode/reservoir portion. To increase the resemblance between anode and cathode, both electrodes would preferably be composite mixtures of silver and silver chloride instead of pure silver and silver chloride. FIG. 8 shows such a system, for example, in which the narcotic analgesic is present in the anodic compartment and the antagonist is present in the cathodic compartment. In FIG. 8, the anode/reservoir portion 150 includes an anodic electrode 152 in contact with a donor side cationic drug reservoir 154 whereas the cathode/reservoir portion 156 includes a cathodic electrode 158 in contact with a counter side antagonist reservoir 157. The anode/reservoir portion 150 and cathode/reservoir portion 156 can be spaced apart in a housing setup similar to that shown in FIG. 2.

More preferably the antagonist is not directly in contact with the skin during normal use but is separated from a skin-contacting gel by an anionic exchange membrane. FIG. 9 shows such an embodiment, in which the cathode/reservoir portion 160 includes an anion exchange membrane 162 separating an antagonist reservoir 164 from a reservoir 166 containing a buffer or salt. FIG. 10 shows yet another alternative embodiment in which, an anionic exchange membrane 168 is also present in the anodic side separating a drug reservoir 170 from a reservoir 172 that contains a halide source, buffer, and the like, to increase the similarity between anode and cathode.

In an alternative embodiment, to increase further the difficulty of identification of the anodic compartment, the anodic compartment is composed of a material that looks like, or is, an integral part of the lower housing of the electrotransport system of FIG. 1. The agonist drug is located in the anodic compartment and the antagonist is not present in an electrode compartment but in some other part of the system such as the circuit board housing. More preferably the antagonist is located in a part that is not directly accessible to the patient. The antagonist can be present as a coating or as a compressed pellet or any other formulation.

In an alternative embodiment, the antagonist is present in the packaging instead of the electrotransport system.

The barrier against antagonist release substantially prevents release of the antagonist from the system upon securing the system to a human patient for a period of up to a day or more; substantially minimizing skin absorption of the antagonist during normal use; and provides release of the antagonist with the agonist when the system is subject to abuse, e.g. upon ingestion or substantial immersion of the donor reservoir portion of the system in the solvent. The barrier allows the ingress of water/solvent in to the antagonist reservoir, thus permitting the release of an antagonist with the agonist when the system is subject to abuse. The antagonist release controlling means include physical means such as a membrane, a film, a coating, a sheet, a deposit. In certain embodiments, the barrier to antagonist release is incorporated within an antagonist reservoir where the rate of release is governed by the osmotic bursting mechanism cited in Gale, et al. The release rate of the antagonist is controlled by factors such as the amount of antagonist within the antagonist reservoir, the antagonist particle size, antagonist salt osmotic pressure, and physical characteristics of the polymer matrix of the antagonist reservoir.

The barrier separating the antagonist from the drug, for example, an ion exchange membrane, can have a thickness of 0.01 mm to about 2 mm, preferably about 0.05 mm to about 1 mm, and even more preferably about 0.1 mm to about 0.5 mm.

As previously mentioned, in certain embodiments the antagonist reservoir includes the antagonist in a multiparticulate form, wherein each particle is individually coated with a polymeric material that substantially prevents release of the antagonist, wherein the polymeric material is preferably a thermoformable material. In additional embodiments, the antagonist reservoir includes beads coated with the antagonist, wherein the beads may be formed from glass or an inert or non-dissolvable polymer, and further wherein the coated beads are optionally coated with or dispersed in a polymeric material which substantially prevents release of the antagonist, wherein the polymeric material is preferably a thermoformable material. Preferably such antagonist-coated particulates are used in conjunction with an ion exchange barrier separating the drug reservoir from the antagonist reservoir. The beads may be in any shape, size or form, but are preferably small sized, preferably less than 10 microns. Examples of an inert or non-dissolvable polymer include, but are not limited to polymethylmethacrylate, polycarbonate and polystyrene.

Preferably, the polymeric material for encapsulating antagonist has a low melting point to allow processing of the antagonist in solid phase and to prevent degradation of the antagonist. Examples of a polymeric material which substantially prevents release of the antagonist include, but are not limited to, polyethylene, polyoctene, polyvinyl acetate, polymethyl acrylate, polymethyl acrylate, polyethyl acrylate, polystyrene polymers and copolymers and mixtures thereof; polystyrene copolymers such as styrenic block copolymers (SIS, SBS, SEBS), ethylene copolymers such as polyethyleneoctene copolymers, ethylene-vinyl acetate copolymer (EVA), ethylenemethyl acrylate copolymers (EMA), ethylene-acrylic acid copolymer, ethylene-ethylacrylate copolymer, and the like, and combinations thereof.

In additional embodiments, the antagonist is complexed with an ionic resin. Examples of ionic resins include, but are not limited to sulfonated polystyrene resins, and the like. Preferably the resin contains a sulfonic acid functionality which when neutralized with the antagonist base forms the sulfonate salt of the antagonist.

The antagonist reservoir includes an amount of the antagonist sufficient to counter the drug (say, analgesic) and euphoric effects of the analgesic when the transdermal analgesic system is abused. The concentration of the agonist (drug) in the agonist reservoir preferably is between 0.05 wt % and 20 wt %, more preferably between 0.1 wt % and 10 wt %. Similarly, the concentration of the antagonist in the antagonist reservoir is preferably between 0.05 wt % and 20 wt %, more preferably between 0.1 wt % and 10 wt %.

When hydrated, the antagonist reservoir can have a thickness of about 0.02 mm to about 2 mm, preferably 0.05 to about 1 mm, and more preferably 0.1 to about 0.5 mm.

In additional embodiments, the drug reservoir may optionally contain additional components such as, additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art, provided that such materials are present below saturation concentration in the reservoir. Such materials can be included by on skilled in the art.

Preferably, a drug delivery system of the present invention would include instruction of use material that gives a description on the how the electrotransport system is to be used, that the system contains antagonist to the drug, and that the antagonist would be released if the system is subject to abuse. Such a description may be presented as an insert that accompanies the device, printed on the surface of the device, printed on the package containing the device, or provided electronically to be displayed when called out, e.g. in a database, in a computer, etc. This description would provide a warning or serve as deterrent to a potential abuser, who will then be more likely to try to find an alternative rather than risking being exposed to the antagonist.

### Administration of the Drug

The present invention provides an electrotransport transdermal drug delivery system having reduced potential for abuse, without diminishing the therapeutic or beneficial effects of the analgesic when the system is applied to the skin.

The electrotransport transdermal drug systems are made as illustrated by examples as follows. The antagonist reservoir and the analgesic reservoirs are made according to known methodology, as described in greater detail below. The Examples below can be carried out to demonstrate sufficient analgesic effect in normal use with substantially negligent antagonist release. However, when the systems in the Examples are treated with a process to mimic abuse, the systems will release the antagonist at a rate that is sufficient to reduce abuse potential.

### EXAMPLE 1

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, where the pH is adjusted to pH 5 using NaOH; 2) an antagonist reservoir having 20 wt % polyvinyl alcohol, 2 wt % naltrexone hydrochloride, and 0.2 wt % polacrilin (IRP-64) in water; the pH is adjusted to pH 5 using NaOH; 3) an anionic exchange membrane (strong base anionic functionality, 0.2 mm thick) weakened in discrete areas (the weakened areas having a thickness of 0.02 mm, representing less than 0.1% of the total membrane area and organized as doted lines separating the membrane in 4 quadrants), and separating the antagonist reservoir from the analgesic reservoir; and 4) a silver anode, pierced with three holes (1 mm in diameter), and contacting the antagonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; and 2) a silver chloride/carbon black/PIB (polyisobutylene) composite cathode contacting the cathode reservoir. The reservoir gels (i.e. the anodic gels and the cathodic gel) sizes are each approximately 0.600 mL and having a skin contacting surface area of about 3 cm². The electrodes are connected to an electrical power source which, when connected, supplies a constant level of electric current in the range of 0.17 mA or 0.057 mA/cm².

### EXAMPLE 2

An electrotransport system similar to that illustrated in FIG. 1 is made. The anode compartment includes 1) an agonist reservoir having 20 wt % polyvinyl alcohol, and 2 wt % fentanyl citrate in water at pH 4; 2) an antagonist reservoir having 20 wt % polyvinyl alcohol, 2 wt % naltrexone HCl, and 0.2 wt % polacrilin (IRP-64) in water, the pH being adjusted to pH 4; 3) an anionic exchange membrane (strong base anionic functionality, 0.2 mm thick) weakened in discrete areas (the weakened areas having a thickness of 0.02 mm, representing less than 0.1% of the total membrane area and organized as doted lines separating the membrane in 4 quadrants), and separating the antagonist reservoir from the analgesic reservoir; and 4) a silver anode, pierced with three holes (1 mm in diameter), and contacting the antagonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water, where the pH is adjusted to pH 4.5 using NaOH or HCl; 2) a silver chloride/carbon black/PIB composite cathode contacting the cathode reservoir. The reservoir gels (i.e. the anodic gels and the cathodic gel) sizes are each approximately 0.600 mL and have a skin contacting surface area of about 3 cm². The electrodes are connected to an electrical power source which, when connected, supplies a constant level of electric current in the range of 0.17 mA or 0.057 mA/cm².

### EXAMPLE 3

An electrotransport system similar to that in FIG. 1 is made. The anode compartment includes 1) an agonist reservoir having 20 wt % polyvinyl alcohol, 2 wt % fentanyl HCl, and 2 wt % His-Glu dipeptide in water at pH 5.2; 2) an antagonist reservoir including 20 wt % polyvinyl alcohol 2 wt % naltrexone HCl, and 2 wt % His-Glu dipeptide in water at pH 5.2; 3) an anionic exchange membrane (strong base anionic functionality, 0.2 mm thick) weakened in discrete areas (the weakened areas having a thickness of 0.02 mm, representing less than 0.1% of the total membrane area and organized as doted lines separating the membrane in 4 quadrants), and separating the antagonist reservoir from the analgesic reservoir; and 4) a silver anode, pierced with three holes (1 mm in diameter), and contacting the antagonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water, where the pH is adjusted to pH 4.5 using NaOH or HCl; 2) a silver chloride/carbon black/PIB composite cathode contacting the cathode reservoir. The reservoir gels (i.e. the anodic gels and the cathodic gel) sizes are each approximately 0.600 mL and have a skin contacting surface area of about 3 cm². The electrodes are connected to an electrical power source which, when connected, supplies a constant level in the range of electric current of 0.17 mA or 0.057 mA/cm².

### EXAMPLE 4

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, the pH is adjusted to pH 5 using NaOH; and 2) a silver anode contacting the agonist reservoir. The cathode compartment includes 1) an antagonist reservoir having 20 wt % polyvinyl alcohol, 2 wt % naltrexone citrate at pH 4; and 2) a silver chloride/carbon black/PIB composite cathode contacting the antagonist reservoir. The reservoir gels (i.e. both the anodic and cathodic gels) sizes are each approximately 0.600 mL and have a skin contacting surface area of about 3 cm². The electrodes are connected to an electrical power source which, when connected, supplies a constant level of electric current in the range of 0.17 mA or 0.057 mA/cm².

### EXAMPLE 5

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, where the pH is adjusted to pH 5 using NaOH; and 2) a silver anode contacting the agonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; and 2) an antagonist reservoir includes 20 wt % polyvinyl alcohol, 2 wt % naltrexone hydrochloride, and 0.2 wt % polacrilin (IRP-64) in water, where the pH is adjusted to pH 4.5; 3) an anionic exchange membrane (strong base anionic functionality, 0.2 mm thick) weakened in discrete areas (the weakened areas having a thickness of 0.02 mm, representing less than 0.1% of the total membrane area and organized as doted lines separating the membrane in 4 quadrants), and separating the antagonist reservoir from the analgesic reservoir; and 4) a silver chloride/carbon black/PIB composite cathode, pierced with three holes (1 mm in diameter), and contacting the antagonist reservoir. The reservoir gels (i.e. the anodic gel and the cathodic gels) sizes are each approximately 0.600 mL and have a skin contacting surface area of about 3 cm². The electrodes are connected to an electrical power source which, when connected, supplies a constant level of electric current in the range of 0.17 mA or 0.057 mA/cm².

### EXAMPLE 6

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, where the pH is adjusted to pH 5 using NaOH; 2) a salt reservoir includes 20 wt % polyvinyl alcohol, 2 wt % NaCl, and 0.2 wt % polacrilin (IRP-64) in water, where the pH is adjusted to pH 5 using NaOH; 3) an anionic exchange membrane (Sybron Chemicals Inc.) separating the salt reservoir from the analgesic reservoir; and 4) a silver anode contacting the salt reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; 2) an antagonist reservoir includes 20 wt % polyvinyl alcohol, 2 wt % naltrexone hydrochloride, and 0.2 wt % polacrilin (IRP-64) in water, where the pH is adjusted to pH 4.5; 3) an anionic exchange membrane (strong base anionic functionality, 0.2 mm thick) weakened in discrete areas (the weakened areas having a thickness of 0.02 mm, representing less than 0.1% of the total membrane area and organized as doted lines separating the membrane in 4 quadrants), and separating the antagonist reservoir from the analgesic reservoir; and 4) a silver chloride/carbon black/PIB composite cathode a silver anode, pierced with several small holes, and contacting the antagonist reservoir. The reservoir gels (ie, the anodic gels and the cathodic gels) sizes are each approximately 0.600 mL and have a skin contacting surface area of about 3 cm². The electrodes are connected to an electrical power source which, when connected, supplies a constant level of electric current in the range of 0.17 mA or 0.057 mA/cm².

### EXAMPLE 7

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, where the pH is adjusted to pH 5 using NaOH; and 2) a silver anode contacting the agonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; and 2) a silver chloride/carbon black/PIB composite cathode contacting the cathode reservoir. The reservoir gels (ie, both the anodic and cathodic gels) sizes are each approximately 0.600 mL and have a skin contacting surface area of about 3 cm². The electrodes are connected to an electrical power source which, when connected, supplies a constant level of electric current in the range of 0.17 mA or 0.057 mA/cm². A formulation (0.2 g) including 10 wt % Naltrexone hydrochloride, 20 wt % hydroxypropylmethyl cellulose (HPMC), and 10 wt % glycerol in water is applied as a thin film to the inside of the circuit board housing and dried prior to assembly of the system.

### EXAMPLE 8

An electrotransport system similar to that illustrated in FIG. 1 is made. The anode compartment includes 1) an agonist reservoir having 20 wt % polyvinyl alcohol, 2 wt % fentanyl hydrochloride, 0.2 wt % polacrilin (IRP-64) in water, and 1 wt % naltrexone hydrochloride microencapsulated in a polymeric resin presenting strong base anionic functionality, where the pH is adjusted to pH 5 using NaOH; and 2) a silver anode contacting the agonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; and 2) a silver chloride/carbon black/PIB composite cathode contacting the cathode reservoir. The reservoir gels (ie, both the anodic and cathodic gels) sizes are each approximately 0.600 mL and have a skin contacting surface area of about 3 cm². The electrodes are connected to an electrical power source which, when connected, supplies a constant level of electric current in the range of 0.17 mA or 0.057 mA/cm².

### EXAMPLE 9

An electrotransport system similar to that illustrated in FIG. 1 is made. The anode compartment includes 1) an agonist reservoir having 20 wt % polyvinyl alcohol, 2 wt % fentanyl hydrochloride, and 0.2 wt % polacrilin (IRP-64) in water, where the pH is adjusted to pH 5 using NaOH; 2) an antagonist reservoir having 20 wt % polyvinyl alcohol, 2 wt % naltrexone hydrochloride, and 0.2 wt % polacrilin (IRP-64) in water; the pH is adjusted to pH 5 using NaOH; 3) an anionic exchange membrane (strong base anionic functionality, 0.2 mm thick), and separating the antagonist reservoir from the analgesic reservoir; and 4) a silver anode, and contacting the antagonist reservoir. The cathode compartment includes 1) a cathode reservoir having 20 wt % polyvinyl alcohol, 0.1 wt % sodium chloride, 0.4 wt % citric acid trisodium salt, 0.2 wt % citric acid, and 0.2 wt % cetylpyrridinium chloride in water at pH 4.5; and 2) a silver chloride/carbon black/PIB composite cathode contacting the cathode reservoir. The reservoir gels (i.e. the anodic gels and the cathodic gel) sizes are each approximately 0.600 mL and having a skin contacting surface area of about 3 cm². The electrodes are connected to an electrical power source which, when connected, supplies a constant level of electric current in the range of 0.17 mA or 0.057 mA/cm².

### EXAMPLE 10

Experiments are performed in vivo in groups of six Caucasian male volunteers, ages 25 to 35 years. The electrotransport systems of Examples 1-9 are applied to and removed from the upper arms of the subjects. The application site is wiped with alcohol prior to system application. Following system application, the circuit is connected (activated) for 10 min every 4 hours for a total of 16 hours and 20 min. Blood samples (10 mL) are taken from each of the patients immediately prior to application of the systems (time 0 hour) and at every hour for 17 hours after system application. Plasma concentrations of fentanyl and naltrexone are determined by LC/MS. Dose delivered is extrapolated from AUC (area under the curve) calculation comparatively to the AUC following intravenous infusion of fentanyl or naltrexone in the same subjects in a different experiment. Results will demonstrate delivery of a nominal 40 μg dose of fentanyl (base equivalent) per every 20 min activation period. No detectable naltrexone delivery (limit of detection of a nominal 5 μg dose) will be observed.

### EXAMPLE 11

The electrotransport systems made according to Examples 1 through 9 are used to study the release of naltrexone and fentanyl from the system upon immersion in water at ambient temperature, i.e. room temperature. The complete transdermal analgesic systems are immersed in 300 mL of distilled water. After selected time intervals, the systems are moved to fresh extraction media. This operation is repeated for a total time of 24 hours. Naltrexone and fentanyl concentrations in the extraction medium are measured by HPLC. By 24 hour, release of fentanyl is almost complete. Significant amounts of naltrexone (i.e., greater than 20% of the dose) will be found to have been released from the systems described in examples 1 through 7.

### EXAMPLE 12

The anodic and cathodic reservoir portions of the electrotransport systems made according to Examples 1 through 8 are used to study the release of naltrexone and fentanyl upon immersion in water at ambient temperature, i.e. room temperature. The anodic and cathodic reservoirs are isolated from each system and immersed together in 50 mL of distilled water. After selected time intervals, the reservoirs are moved to fresh extraction media. This operation is repeated for a total time of 24 hours. Naltrexone and fentanyl concentrations in the extraction medium are measured by HPLC. By 24 hours, release of fentanyl and naltrexone is almost complete from the systems described in examples 1 through 6.

### EXAMPLE 13

The anodic and cathodic reservoir portions of the electrotransport systems made according to Examples 8 are used to study the release of naltrexone and fentanyl following an attempt to mimic a chewing mode of abuse. The anodic and cathodic reservoirs are placed in an agate mortar and are compressed repetitively against the bowl with an agate pestle. The anodic and cathodic reservoirs are subsequently immersed together in 50 mL of distilled water. After 24 hours incubation, naltrexone and fentanyl concentrations in the extraction medium are measured by HPLC. By 24 hours, release of fentanyl is almost complete. A significant amount of naltrexone (i.e. greater than 20% of the dose) will be found to have been released from the reservoirs.

### EXAMPLE 14

The electrotransport systems made according to Examples 1 through 9 as well as a fentanyl electrotransport system containing no antagonist are presented to a group of about 2 dozen males and females convicted abusers that participate voluntarily in the survey. In addition, a passive fentanyl transdermal delivery system (Duragesic®) and an illicit drug substances (crack cocaine) are shown to the volunteers. When asked to rank which system/substance they would prefer to abuse, the majority of volunteers would prefer the illicit drug substance. The Duragesic system is less preferred, followed by the electrotransport system containing no antagonist. When asked if they would attempt to abuse any of the systems described in Examples 1 through 8, the majority of volunteers would answer no. Only a small fraction of the surveyed individuals might mention that they would attempt to abuse these systems only if no other alternative was available. This example is to demonstrate that the mere presence of an antagonist in the system is a powerful deterrent against abuse.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope of the present invention. The entire disclosure of each patent, patent application, and publication cited or described in this document is hereby incorporated herein by reference. The embodiments are intended to be illustrative in all respects, rather than restrictive, of the present invention. It is to be understood that various combinations and permutations of various parts and components of the schemes disclosed herein can be implemented by one skilled in the art without departing from the scope of the present invention.

What is claimed is:

1. A transdermal electrotransport system for administering an analgesic through the skin, the system having a reduced potential for abuse of the analgesic, comprising:
(a) electrode for conducting a current to drive analgesic ions of an ionizable analgesic;
(b) an analgesic reservoir comprising the analgesic ions;
(c) an antagonist source comprising an antagonist for said analgesic; and
(d) a barrier, said barrier separating said antagonist source from said analgesic reservoir, said barrier being substantially impermeable to said analgesic ions and to said antagonist, wherein the antagonist is released with the analgesic ions when the system is subject to abuse.

2. The system of claim 1 wherein the barrier is one of an anion exchange barrier separating a cationic antagonist from a cationic analgesic and a cation exchange barrier separating an anionic antagonist from an anionic analgesic.

3. The system of claim 2 wherein the analgesic is a cationic drug and the barrier includes an anionic ion exchange material and the analgesic reservoir is at a pH of from 2 to 6.

4. The system of claim 3 wherein the analgesic is a cationic drug, the barrier includes an anionic ion exchange membrane, the analgesic reservoir is at a pH of from 3 to 5 and the analgesic is selected from a group consisting of salts of fentanyl and analogs thereof.

5. The system of claim 4 wherein the ion exchange membrane does not contact the electrode or skin of a user.

6. The system of claim 3 wherein the barrier is selected from a group consisting of a layer, a membrane, a film, a coating, a sheet, and a deposit on the antagonist reservoir and the barrier has weak area and less weak area, the weak area preferentially breaks before the less weak area when under stress or placed in solvent.

7. The system of claim 3 wherein the antagonist source comprises particulates including the antagonist and the barrier is a coat of the particulate antagonist enveloping the antagonist.

8. The system of claim 3 wherein the antagonist source comprises particulates including the antagonist and the barrier is a coat of the particulates and the particulates including antagonist are dispersed in the analgesic reservoir.

9. The system of claim 3 comprising a reservoir having a halide ion source.

10. The system of claim 3 comprising an antagonist layer including dissolved antagonist solubilized in the antagonist layer or including particulates containing antagonist, the antagonist layer being nearer to skin of a user than the drug reservoir.

11. The system of claim 3, wherein said analgesic reservoir comprises an amount of analgesic sufficient to induce and maintain analgesia in a human patient for a period of at least one day.

12. The system of claim 3 wherein the analgesic is a salt of fentanyl and analogs thereof and said analgesic reservoir comprises a polymer having a hydrogel.

13. The system of claim 3 wherein the analgesic reservoir comprises a salt of fentanyl or a salt of sufentanil and the anion exchange material is an anion exchange membrane disposed between a proximal layer of the analgesic reservoir from a more distal layer of the antagonist reservoir, the anion exchange membrane not contacting either skin or the electrode.

14. The system of claim 3 wherein the analgesic is a salt of fentanyl or an analog thereof and the analog is selected from the group consisting of alfentanil, lofentanil, remifentanil, sufentanil and trefentanil; and the antagonist is selected from the group consisting of naltrexone, methylnaltrexone, naloxone, nalbuphine, nalorphine, nalorphine dinicotinate, nalmefene, nadide, levallorphan, cyclozocine and pharmaceutically acceptable salts thereof.

15. The system of claim 3, wherein the analgesic reservoir comprises a polymeric matrix having about 0.1 wt % to about 10 wt % of the analgesic.

16. The system of claim 3, wherein the analgesic is a salt of fentanyl or an analog thereof and the analog is selected from the group consisting of alfentanil, lofentanil, carfentanil, remifentanil, sufentanil and trefentanil; and the antagonist is selected from the group consisting of amiphenazole, naltrexone, methylnaltrexone, naloxone, nalbuphine, nalorphine, nalorphine dinicotinate, nalmefene, nadide, levallorphan, cyclozocine and pharmaceutically acceptable salts thereof, and wherein the barrier is selected from a group consisting of a layer, a membrane, a film, a coating, a sheet, and a deposit on the antagonist reservoir and the barrier has weak area and less weak area, the weak area preferentially breaks before the less weak area when under stress or placed in solvent.

17. The system of claim 3 comprises an anode portion that includes (a) and (b); and the system further comprising a cathode portion that is visually indistinguishable from said anode portion without referring to electrical connection to the system, said cathode portion including (a) and (b).

18. The system of claim 1 wherein the barrier is a dialysis membrane.

19. The system of claim 1 wherein the barrier is a dialysis membrane with a molecular weight cutoff of 200 Da or less.

20. The system of claim 1 wherein the barrier is a dialysis membrane with a molecular weight cutoff of 100 Da or less.

21. A method for making a transdermal electrotransport system for administering an analgesic through the skin, the system having a reduced potential for abuse of the analgesic, comprising:
(a) providing electrode for conducting a current to drive analgesic ions of an ionizable analgesic;
(b) providing ionic flow path from the electrode to an analgesic reservoir comprising the analgesic ions; and
(c) providing an antagonist source comprising an ionic antagonist for said analgesic such that the antagonist source and the analgesic reservoir are separated by a barrier that is substantially impermeable to said analgesic ions and to said ionic antagonist, wherein the ionic antagonist is released with the analgesic ions when the system is subject to abuse.

22. The method of claim 21, comprising separating the antagonist source and the analgesic reservoir using an anion exchange barrier if the ionic antagonist is cationioc and using a cation exchange barrier if the ionic antagonist is anionic.

23. The method of claim 22, comprising separating the antagonist source and the analgesic reservoir using an anion exchange membrane and adjusting the pH of the analgesic reservoir to a pH of 2 to 6, wherein the analgesic reservoir contains a cationic drug, the method providing in the membrane weak area and less weak area such that the weak area preferentially breaks before the less weak area when subjected to stress or solvent.

24. The method of claim 22, comprising separating the antagonist source and the analgesic reservoir using an anion exchange membrane and adjusting the pH of the analgesic reservoir to a pH of 2 to 6, wherein the analgesic reservoir contains a salt of fentanyl or a salt of fentanyl analog.

25. The method of claim 22, comprising including antagonist in particulates and dispersing the particulates in a layer through which the analgesic can migrate iontophoretically.

26. The method of claim 22, comprising providing a microporous membrane as the barrier.

27. A transdermal electrotransport system for administering an analgesic through the skin, the system having a reduced potential for abuse of the analgesic, comprising:
(a) first electrode-reservoir portion having first electrode for conducting a current to drive an ionic analgesic and an analgesic reservoir comprising the ionic analgesic; and
(b) second electrode-reservoir portion having second electrode of opposite polarity to the first electrode, an antagonist source comprising an antagonist for said ionic analgesic and a barrier separating said antagonist source from skin of a user, said barrier being substantially impermeable to said ionic analgesic and to said antagonist, wherein the antagonist is released with the ionic analgesic when the system is subject to abuse; wherein the first electrode-reservoir portion and the second electrode-reservoir portion are visually indistinguishable from each other without referring to electrical connections thereto.

28. The system of claim 27 wherein the ionic analgesic is a cationic drug, the antagonist source having cationic antagonist ions and the barrier includes an anion ion exchange layer and the analgesic reservoir is at a pH of from 2 to 6.

29. The system of claim 28 wherein the ionic analgesic is a cationic drug, the barrier includes an anionic ion exchange membrane, the analgesic reservoir is at a pH of from 3 to 5 and the analgesic is selected from a group consisting of a salt of fentanyl and analogs thereof.

30. The system of claim 28 wherein the barrier is an ion exchange membrane and does not contact the electrode or skin of a user.

31. The system of claim 28 wherein the barrier is selected from a group consisting of a layer, a membrane, a film, a coating, a sheet, and a deposit on the antagonist reservoir, the barrier having weak area and less weak area.

32. The system of claim 28 wherein the antagonist source comprises particulates including the antagonist and the barrier is a coat of the particulates.

33. A transdermal electrotransport system for administering an analgesic through the skin, the system having a reduced potential for abuse of the analgesic, comprising:
(a) electrode for conducting a current to drive analgesic ions of an analgesic;
(b) an analgesic reservoir comprising the analgesic; and
(c) an antagonist source comprising an ionized antagonist for said analgesic.

34. A method for using a transdermal electrotransport system for administering an analgesic through the skin with a reduced potential for abuse of the analgesic, comprising:
(a) providing a transdermal electrotransport system with electrode for conducting a current to drive analgesic ions of an ionizable analgesic, ionic flow path from the electrode to an analgesic reservoir comprising the analgesic ions, and an antagonist source comprising an ionic antagonist for said analgesic such that the antagonist source and the analgesic reservoir are separated by a barrier layer that is substantially impermeable to said analgesic ions and to said ionic antagonist, wherein the ionic antagonist is released with the analgesic ions when the system is subject to abuse; and
(b) providing a readable instruction on the operation of the electrotransport system and a warning that the system has an antagonist that would be released upon abuse of the system.
